(19) United States
(12) Patent Application Publication
(10) Pub. No.: US 2011/0131854 A1
(43) Pub. Date: Jun. 9, 2011

(54) SHEET OF WRISTBANDS

(75) Inventor: Bryan M. Waltersdorf, Oconomowoc, WI (US)

(73) Assignee: WRISTBAND RESOURCES, INC., Brookfield, WI (US)

(21) Appl. No.: 12/634,538

(22) Filed: Dec. 9, 2009

(51) Int. Cl. A61B 5/117; A44C 5/00

(52) U.S. Cl. 40/633

(57) ABSTRACT

A sheet of wristbands comprising a plurality of wristband straps, each including opposed side edges. A side edge of each strap is coupled to a side edge of an adjacent strap to form a series of side-by-side straps. A first strap defines a first side edge of the series and a second strap defines a second side edge of the series. A closure is positioned on each strap. A first tear strip is coupled to the first side edge, and a second tear strip is coupled to the second side edge. The first tear strip has a first length, and the second tear strip has a second length substantially equal to the first length. It is preferred that the first and second lengths are substantially equal to a length of the wristband straps. At least one wristband strap has an end edge that is exposed. Each tear strip can also have an end edge that is exposed. Preferably, the tear strips each have a width that is narrower than the width of the wristband straps. Each strap can also have tamper cuts, but it is preferred that the strips do not have tamper cuts.

### BACKGROUND

The present invention generally relates to wristbands, and specifically to wristbands that are formed in a sheet.

Wristbands are commonly used as a means for identifying an individual. For example, wristbands can be used to identify individuals that are authorized to be in a certain location (e.g., at a concert or a park) or to consume certain food or beverages (e.g., at a party), such as alcoholic beverages. Wristbands are commonly of a certain color or imprinted pattern to facilitate visual identification at a distance.

### SUMMARY

The present invention provides a sheet of wristbands comprising a plurality of wristband straps, each strap including opposed side edges. A side edge of each strap is detachably coupled (e.g., perforated or scored) to a side edge of an adjacent strap to form a series of side-by-side straps. A first strap defines a first side edge of the series of straps and a second strap defines a second side edge of the series of straps. A closure (e.g., adhesive with a removable liner) is positioned on each strap. A first tear strip is positioned adjacent to the first strap and detachably coupled (e.g., perforated or scored) to the first side edge, and a second tear strip is positioned adjacent to the second strap and detachably coupled (e.g., perforated or scored) to the second side edge.

In one aspect, the first tear strip has a first length, and the second tear strip has a second length substantially equal to the length of the first length. In this embodiment, it is preferred that the first and second lengths are substantially the same as a length of at least one of the wristband straps (e.g., the same as the length of all of the straps).

Preferably, the tear strips each have a width that is narrower than the width of the wristband straps. For example, the tear strips can have a width that is less than 75% and at least 50% (e.g., about 66%) of the width of the wristband straps. Each strap can also have tamper cuts, but it is preferred that the strips do not have tamper cuts.

In another aspect of the invention, at least one wristband strap has an end edge that is exposed. In one embodiment, all of the wristband straps have an end edge that is exposed, and they each can have two end edges that are exposed. Each tear strip can also have an end edge that is exposed, and preferably each has two end edges that are exposed. The exposed end edges can be created with a clean cut (e.g., without a perforation), which creates a smoother end edge that can be aesthetically more attractive and more comfortable when contacting a user's skin.

Other aspects of the invention will become apparent by consideration of the detailed description and accompanying drawings.

### BRIEF DESCRIPTION OF THE DRAWINGS

FIG. 1 is a front view of a sheet of wristbands embodying the present invention.

FIG. 2 is a front view of the sheet of wristbands of FIG. 1 with two tear strips removed.

FIG. 3 is a front view of a single wristband strap removed from the sheet of wristbands of FIG. 1.

FIG. 4 is a side view of the wristband strap of FIG. 3.

FIG. 5 is a schematic of a production line for manufacturing the sheet of wristbands of FIG. 1.

FIG. 6 is a perspective view of the sheet of wristbands of FIG. 1 being fed to a printer.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

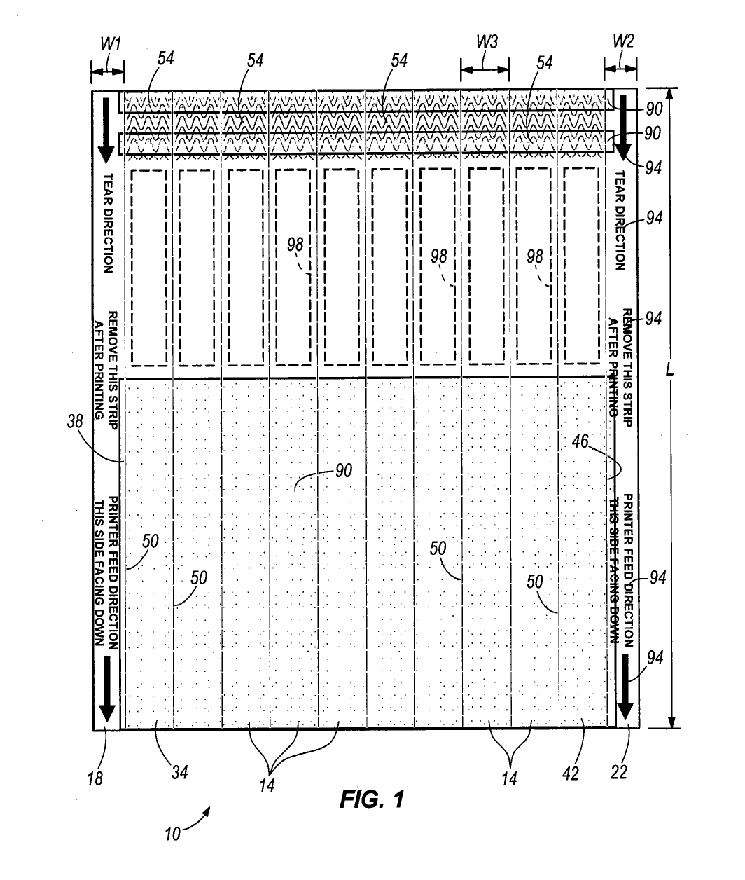

FIG. 1 shows a sheet of wristbands 10 that includes multiple wristband straps 14, a first tear strip 18, a second tear strip 22, a closure in the form of an adhesive 26 (see FIG. 4), and a liner 30 (see FIG. 4) positioned at one end of the sheet of wristbands 10. The illustrated sheet of wristbands 10 includes ten wristband straps 14, each including opposed side edges. At least one side edge of each strap 14 is detachably coupled to a side edge of an adjacent wristband strap to form a series of side-by-side wristband straps 14. In other constructions, the sheet of wristbands 10 may include more than ten or less than ten wristband straps 14, as desired.

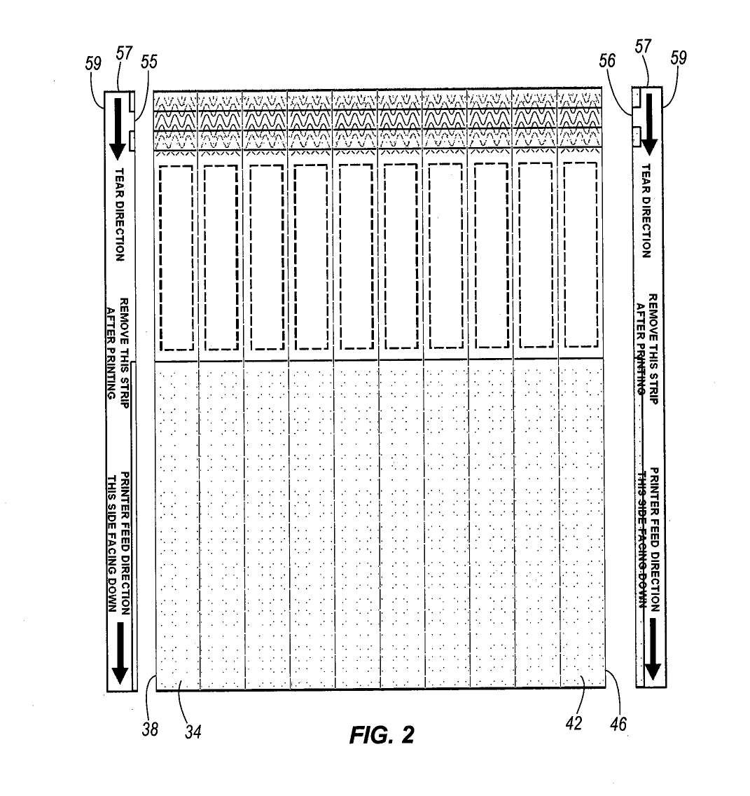

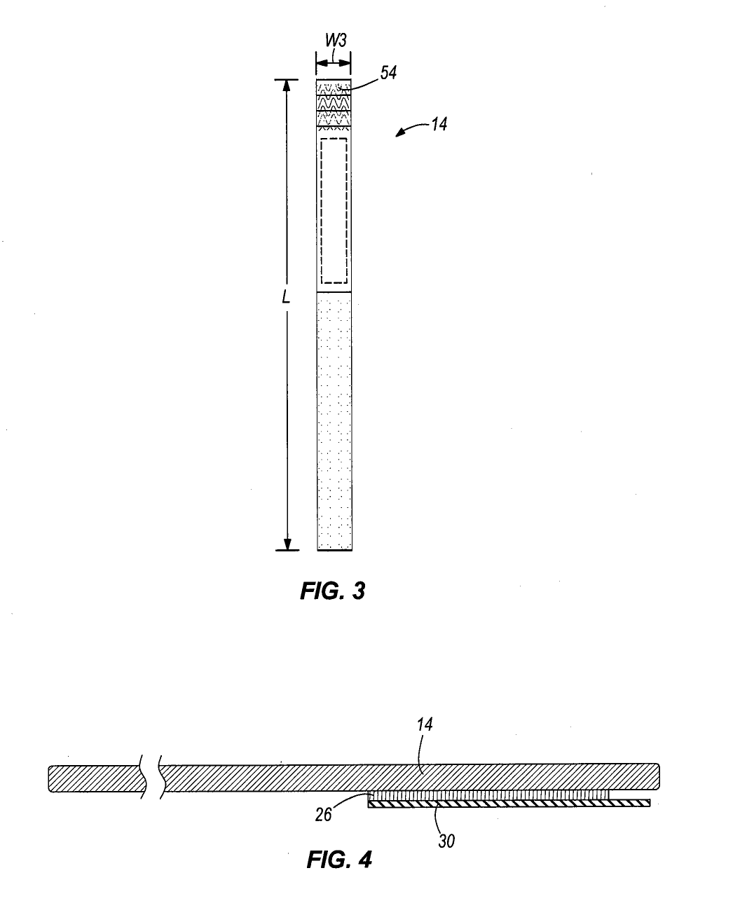

A first strap 34, shown on the left in FIG. 2, defines a first side edge 38 of the series of wristband straps 14 and a second strap 42, shown on the right in FIG. 2, defines a second side edge 46 of the series of wristband straps 14. The remaining wristband straps 14 are detachably coupled between the first strap 34 and the second strap 42. The illustrated wristband straps 14 are detachably coupled together via perforations 50. In other words, the sheet of wristbands 10 includes a detachable coupling in the form of a perforation 50 in between each wristband strap 14 to allow the wristband straps 14 to be separated (e.g., torn apart). FIG. 3 shows an individual wristband strap 14 removed from the sheet of wristbands 10. Each wristband strap 14 also includes tamper cuts 54 at one end. In other constructions, the spacing of the perforations 50 may be different or the perforations 50 may be replaced by a score or other appropriate separation mechanism, as desired.

Each individual wristband strap 14 extends substantially the full length L of the sheet of wristbands 10 such that each wristband strap 14 has at least one exposed end edge with respect to the sheet of wristbands 10. In the illustrated embodiment, the length L is 254 mm. The illustrated wristband straps 14 include two end edges that are exposed with respect to the sheet of wristbands 10 such that the length L of the sheet of wristbands 10 is substantially equal to the length of each individual wristband strap 14. All the illustrated wristband straps 14 have substantially the same length. Because the end edges are exposed (i.e., there is no tear strip or other item secured at that location), the end edges can be cut with a complete through-out (i.e., all the way through and completely across each end edge) instead of a perforation cut. The complete through-cut creates a smooth edge (compared to a perforation cut), which can be more comfortable against the skin of the user.

The illustrated first tear strip 18 is positioned on the left side of the sheet of wristbands 10 in FIG. 1 and adjacent to the first strap 34. Referring to FIG. 2, the first tear strip 18 includes a side edge 55 that is detachably coupled to the first side edge 38 of the series of wristband straps 14. The illustrated first tear strip 18 includes a detachable coupling in the form of a perforation 50 or score in between the first tear strip 18 and the first strap 34, and defines a first length that is substantially the same as the length L of the wristband straps 14. FIG. 2 shows the first tear strip 18 removed from the sheet of wristbands 10.

The illustrated second tear strip 22 is positioned on the right side of the sheet of wristbands 10 in FIG. 1 and adjacent to the second strap 42. The second tear strip 22 includes a side edge 56 that is detachably coupled to the second side edge 46 of the series of wristband straps 14. The illustrated second tear strip 22 includes a detachable coupling in the form of a perforation 50 or score in between the second tear strip 22 and the second strap 42, and defines a second length that is substantially the same as the length L of the wristband straps 14 and is substantially equal to the first length. FIG. 2 shows the second tear strip 22 removed from the sheet of wristbands 10.

Both the first tear strip 18 and the second tear strip 22 include an end edge 57 that is exposed and a side edge 59 that is exposed. The first tear strip 18 defines a first width W1 and the second tear strip 22 defines a second width W2. In the illustrated embodiment, the first width W1 is substantially equal to the second width W2 and is about 12.7 millimeters wide. The first width W1 and the second width W2 are both narrower than a strap width W3 of the wristband straps 14 which are about 19 millimeters wide. Neither the illustrated first tear strip 18 nor the illustrated second tear strip 22 includes tamper cuts 54.

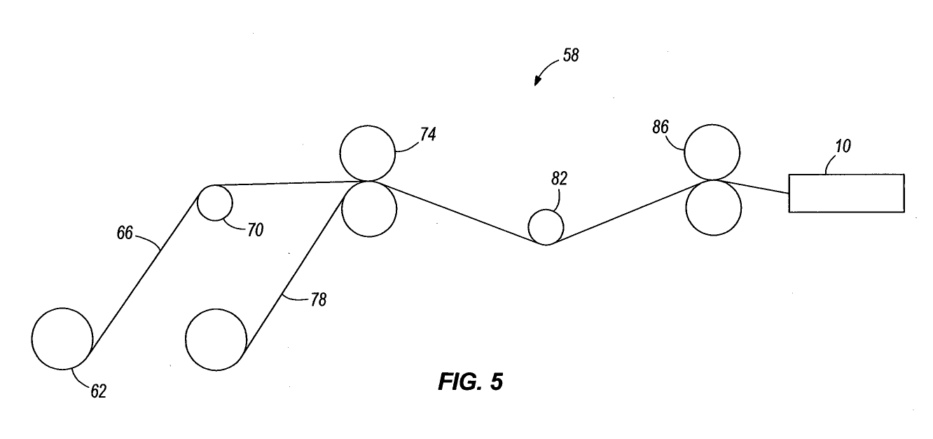

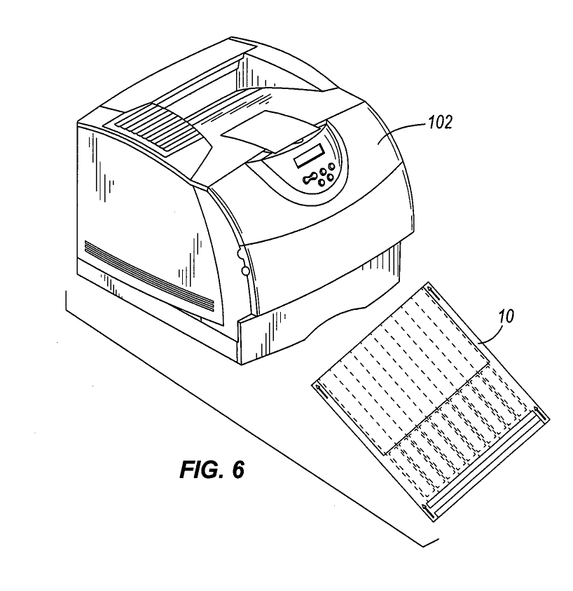

The above-described sheet of wristbands 10 can be manufactured with a machine 58 that is schematically illustrated in FIG. 5. The machine 58 starts with a roll of material 62 that is pulled off in a web 66 and passed over a roller 70. The web 66 is moved into the nip of laminating rolls 74, and a release liner tape 78 including the adhesive 26 and the liner 30 is brought into contact with the web 66. The web 66 is then passed to an idler roller 82 and a sheet cutter 86, where the web 66 is cut into sheets of wristbands 10. The illustrated sheet cutter 86 also cuts the perforations 50 and the tamper cuts 54 into each sheet of wristbands 10. In other embodiments, additional actions may occur during production, as desired. For example, a printing operation, other cutting actions, stamping action, or another action could be implemented, as desired.

After the sheet of wristbands 10 is produced, printed sections may be added (see FIG. 1) including solid color blocks 90 and directions 94 including text, arrows, or other prompts. Other images or blocks of color could be printed, as desired, to provide a predetermined pattern or appearance. Alternatively, the printing may be done during the production in the machine 58 shown in FIG. 5 or in a step intermediate to any of the production steps. Various printing methods may be used.

The illustrated sheets of wristbands also include image portions 98 provided on each individual wristband strap 14. As shown in FIG. 6, the sheets of wristbands 10 may be provided to a printer 102 to print a desired image in the image portions 98. The image portions 98 may be arranged differently than shown. The illustrated printer 102 is a thermal transfer printer, although other printer types are contemplated. The printer 102 may be utilized by the producer of the sheet of wristbands 10, a distributor, an agent of the producer, a printing company, the end user, or another entity, as desired.

The sheet of wristbands 10 can be made from any of a variety of appropriate materials, such as paper, plastic, Tyvek® (a trademark of E.I. DuPont de Nemours), or a composite. Further, the wristband straps 14 can be sized and shaped as desired.

In operation, when a wristband strap 14 is separated from the sheet of wristbands 10, it is designed to wrap around and secure to an item or person (e.g., a person's wrist or ankle). The liner 30 covers the adhesive 26 when produced and before use. When the liner 30 is removed the adhesive 26 is free to secure the wristband strap 14 into a loop. The adhesive 26 is positioned near one end of the wristband strap 14 and provides a mechanism for securing that end of the wristband strap 14 to another portion of the wristband strap 14 in order to form the loop around an object. The illustrated adhesive 26 covers the full width W3 of the wristband strap 14 and is shaped in a rectangular configuration. The adhesive 26 could be any suitable adhesive, such as a modified acrylic composition, a rubber-based adhesive, and the like.

Various features and advantages of the invention are set forth in the following claims.

What is claimed is:

1. A sheet of wristbands comprising:
a plurality of wristband straps, each strap including opposed side edges, wherein a side edge of each strap is detachably coupled to a side edge of an adjacent strap to form a series of side-by-side straps, wherein a first strap defines a first side edge of the series of straps and a second strap defines a second side edge of the series of straps;
a closure positioned on each strap; and
a first tear strip positioned adjacent to the first strap and detachably coupled to the first side edge, the first tear strip having a first length; and
a second tear strip positioned adjacent to the second strap and detachably coupled to the second side edge, the second tear strip having a second length substantially equal to the first length, and wherein the first and second lengths are substantially the same as a length of at least one of the wristband straps.

2. A sheet of wristbands as defined in claim 1, wherein all of the wristband straps have substantially the same length.

3. A sheet of wristbands as defined in claim 1, wherein at least one wristband strap has an end edge that is exposed.

4. A sheet of wristbands as defined in claim 3, wherein each wristband strap has an edge that is exposed.

5. A sheet of wristbands as defined in claim 4, wherein the tear strips each have end edges that are exposed.

6. A sheet of wristbands as defined in claim 1, wherein the tear strips each have a width that is narrower than a width of at least one of the wristband straps.

7. A sheet of wristbands as defined in claim 6, wherein the width of the tear strips is less than 75% of the width of wristband straps

8. A sheet of wristbands as defined in claim 7, wherein the width of the tear strips is at least 50% of the width of wristband straps.

9. A sheet of wristbands as defined in claim 1, wherein a detachable coupling between straps comprises a perforation.

10. A sheet of wristbands as defined in claim 1, wherein a detachable coupling between straps and strips comprises a perforation.

11. A sheet of wristbands as defined in claim 1, wherein each strap includes tamper cuts, but strips do not include tamper cuts.

12. A sheet of wristbands comprising:
a plurality of wristband straps, each strap including opposed side edges, wherein a side edge of each strap is detachably coupled to a side edge of an adjacent strap to form a series of side-by-side straps, wherein a first strap defines a first side edge of the series of straps and a second strap defines a second side edge of the series of straps, and wherein at least one wristband strap has an end edge that is exposed;
a closure positioned on each strap; and
a first tear strip positioned adjacent to the first strap and detachably coupled to the first side edge; and
a second tear strip positioned adjacent to the second strap and detachably coupled to the second side edge.

13. A sheet of wristbands as defined in claim 12, wherein each wristband strap has an edge that is exposed.

14. A sheet of wristbands as defined in claim 13, wherein the tear strips each have an end edge that is exposed.

15. A sheet of wristbands as defined in claim 12, wherein the tear strips each have a width that is narrower than a width of at least one of the wristband straps.

16. A sheet of wristbands as defined in claim 15, wherein the width of the tear strips is less than 75% of the width of wristband straps

17. A sheet of wristbands as defined in claim 16, wherein the width of the tear strips is at least 50% of the width of wristband straps.

18. A sheet of wristbands as defined in claim 12, wherein a detachable coupling between straps comprises a perforation.

19. A sheet of wristbands as defined in claim 12, wherein a detachable coupling between straps and strips comprises a perforation.

20. A sheet of wristbands as defined in claim 12, wherein each strap includes tamper cuts, but strips do not include tamper cuts.
